(19) United States
(12) Patent Application Publication
(10) Pub. No.: US 2021/0081587 A1
(43) Pub. Date: Mar. 18, 2021

(54) METHOD FOR BUILDING PREDICTIVE MODEL OF MICROORGANISM-DERIVED DISSOLVED ORGANIC NITROGEN IN WASTEWATER

(71) Applicant: Nanjing University, Nanjing (CN)

(72) Inventors: Haidong HU, Nanjing (CN); Kewei LIAO, Nanjing (CN); Hongqiang REN, Nanjing (CN)

(21) Appl. No.: 17/024,656

(22) Filed: Sep. 17, 2020

Related U.S. Application Data
(63) Continuation-in-part of application No. PCT/CN2019/119772, filed on Nov. 20, 2019.

(30) Foreign Application Priority Data
Sep. 12, 2019 (CN) 201910861998.7

(51) Int. Cl. G06F 30/27; C02F 3/00

(52) U.S. Cl. CPC G06F 30/27; C02F 2101/38; C02F 3/006

(57) ABSTRACT

A method for building a predictive model of mDON in wastewater, including a) acquiring a kinetics associated with production and consumption of a mDON of an activated sludge system, and importing a kinetic expression of the mDON into a conventional activated sludge model No. 1 (ASM1) to build a kinetic equation for the mDON; b) inputting component variables, parameter variables, model matrices, process rate equation and operating parameters of a predictive model into a simulation software AquaSim to build an ASM-mDON model; c) inputting initial values of the component variables and the parameter variables into the simulation software AquaSim for model initialization; d) acquiring initial mDON kinetic and sensitivity analysis results, selecting corresponding parameters, calibrating kinetic and stoichiometric parameters of the ASM-mDON model using a parameter estimation function of the simulation software AquaSim; and e) replacing the initial values of the ASM-mDON model with optimal values obtained in d).

### CROSS-REFERENCE TO RELATED APPLICATIONS

This application is a continuation-in-part of International Patent Application No. PCT/CN2019/119772 with an international filing date of Nov. 20, 2019, designating the United States, now pending, and further claims foreign priority benefits to Chinese Patent Application No. 201910861998.7 filed Sep. 12, 2019. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

The disclosure relates to the field of wastewater treatment, and more particularly, to a method for building a predictive model of microorganism-derived dissolved organic nitrogen (mDON) in wastewater and to application thereof.

The effluent of the municipal wastewater treatment plants includes dissolved organic nitrogen (DON). In general, the DON includes influent-derived dissolved organic nitrogen (inDON) which is non-degradable and microorganism-derived dissolved organic nitrogen produced in the biological sewage treatment process. Compared with inDON, mDON produced in the wastewater treatment process is more easily affected by the process parameters and conditions, and the composition and properties of mDON are closely related to the growth and metabolism of microorganisms in the biological treatment process.

Although the mDON in the sewage treatment plant has attracted increasing attention, there is no direct method to determine mDON in the sewage treatment plant.

### SUMMARY

The disclosure provides a method for building a predictive model of microorganism-derived dissolved organic nitrogen (mDON) in wastewater. Specifically, based on the operating parameters of an activated sludge process, the component concentrations, and the kinetic and stoichiometric parameters of the influent of a sewage plant, an activated sludge model (ASM)-mDON predictive model is built.

Provided is a method for building a predictive model of mDON in wastewater, the method comprising:
a) acquiring a kinetics associated with production and consumption of a mDON of an activated sludge system, and importing a kinetic expression of the mDON into a conventional activated sludge model No. 1 (ASM1) to build a kinetic equation for the mDON;
b) inputting component variables, parameter variables, model matrices, process rate equation and operating parameters of a predictive model into a simulation software AquaSim to build an ASM-mDON model;
c) inputting initial values of the component variables and the parameter variables into the simulation software AquaSim for model initialization;
d) acquiring initial mDON kinetic and sensitivity analysis results, selecting corresponding parameters, calibrating kinetic and stoichiometric parameters of the ASM-mDON model using a parameter estimation function of the simulation software AquaSim, thereby predicting a concentration of the mDON; and
e) replacing the initial values of the ASM-mDON model with optimal values obtained by the parameter estimation function in d), thereby optimizing the model.

The activated sludge system comprises a fully mixed steady state activated sludge; the activated sludge has a sludge age of 5-30 days, and a concentration of 2000-5000 mg/L.

In c), the initial values of the parameter variables are determined with reference to “Mathematical Model for Activated Sludge”. The kinetic and stoichiometric parameters of the ASM-mDON model are classified for parameter assumption, parameter estimation, or default argument assignment.

In d), the sensitivity analysis uses the absolute-relative sensitivity equation to determine the influence of different values of an independent parameter on the estimation of the mDON.

The ASM-mDON model is used for study of the mDON released by microorganisms in the activated sludge system, and the model comprises:
seven components: heterotrophic bacteria X_{H}, autotrophic bacteria X_{A}, inert particles X₁, nitrate nitrogen S_{NO}, ammonia nitrogen S_{NH}, microorganism-derived dissolved organic nitrogen S_{DON}, dissolved oxygen S_{O};
five reaction processes: the growth process and endogenous respiration process of heterotrophic bacteria using ammonium chloride as a substrate; the growth process and endogenous respiration process of autotrophic bacteria using ammonium chloride as a substrate; and the ammonization process of mDON;
eighteen parameters: maximum specific growth rate \hat{μ}_{H} of heterotrophic bacteria, yield coefficient Y_{H} of heterotrophic bacteria, attenuation coefficient b_{H} of heterotrophic bacteria, half-saturation constant K_{H,NH} for ammonia nitrogen of heterotrophic bacteria, half-saturation constant K_{H,O} for dissolved oxygen of heterotrophic bacteria, maximum specific growth rate \hat{μ}_{A} of autotrophic bacteria, substrate utilization ratio f_{H,DON} of heterotrophic bacteria converting the substrates into the mDON, yield coefficient Y_{A} of autotrophic bacteria, attenuation coefficient b_{A} of autotrophic bacteria, half-saturation constant K_{A,NH} for ammonia nitrogen of autotrophic bacteria, half-saturation constant K_{A,O} for dissolved oxygen of autotrophic bacteria, substrate utilization ratio f_{A,DON} of autotrophic bacteria converting the substrates into the mDON, proportion of nitrogen i_{XB} in the organism, proportion of nitrogen i_{XP} in the product of the organism, substrate utilization ratio f_{NO} of autotrophic bacteria converting the substrates into the nitrate nitrogen, proportion of inert particles f_{I} yielded in the organism, ammonification rate kₐ, and half-saturation constant K_{H,DON} for mDON.

The change rates of the seven components of the ASM-mDON model satisfy with the following formulas:

$$\begin{matrix}{X}_{H} \text{:} \frac{{dX}_{H}}{dt} = {\hat{μ}}_{H} {M}_{H , NH} ( t ) {M}_{H , O} ( t ) {X}_{H} ( t ) - {b}_{H} {M}_{H , O} ( t ) {X}_{H} ( t ) & ( 1 ) \\ {X}_{A} \text{:} \frac{{dX}_{A}}{dt} = {\hat{μ}}_{A} {M}_{A , NH} ( t ) {M}_{A , O} ( t ) {X}_{A} ( t ) - {b}_{A} {M}_{A , O} ( t ) {X}_{A} ( t ) & ( 2 ) \\ {S}_{NH} \text{:} \frac{{dS}_{NH}}{dt} = - ( \frac{{f}_{H , DON}}{{Y}_{H}} + {i}_{XB} ) {\hat{μ}}_{H} {M}_{H , NH} ( t ) {M}_{H , O} ( t ) {X}_{H} ( t ) - ( \frac{{f}_{A , DON} + {f}_{NO}}{{Y}_{A}} + {i}_{XB} ) {\hat{μ}}_{A} {M}_{A , NH} ( t ) {M}_{A , O} ( t ) {X}_{A} ( t ) + {k}_{a} {M}_{H , DON} ( t ) {X}_{H} ( t ) & ( 3 ) \\ {S}_{DON} \text{:} \frac{{dS}_{DON}}{dt} = \frac{{f}_{H , DON}}{{Y}_{H}} {\hat{μ}}_{H} {M}_{H , NH} ( t ) {M}_{H , O} ( t ) {X}_{H} ( t ) + \frac{{f}_{A , DON}}{{Y}_{A}} {\hat{μ}}_{A} {M}_{A , NH} ( t ) {M}_{A , O} ( t ) {X}_{A} ( t ) - {k}_{a} {M}_{H , DON} ( t ) {X}_{H} ( t ) & ( 4 ) \\ {S}_{NO} \text{:} \frac{{dS}_{NO}}{dt} = \frac{{f}_{NO}}{{Y}_{A}} {\hat{μ}}_{A} {M}_{A , NH} ( t ) {M}_{A , O} ( t ) {X}_{A} ( t ) & ( 5 ) \\ {X}_{I} \text{:} \frac{{dX}_{I}}{dt} = {f}_{I} {b}_{H} {M}_{H , O} ( t ) {X}_{H} ( t ) + {f}_{I} {b}_{A} {M}_{A , O} ( t ) {X}_{A} ( t ) & ( 6 ) \\ {S}_{O} \text{:} \frac{{dS}_{O}}{dt} = {k}_{L} α ( {S}_{O}^{*} - {S}_{O} ) - ( 1 - \frac{2.86 {f}_{H , DON}}{{Y}_{H}} ) {\hat{μ}}_{H} {M}_{H , NH} ( t ) {M}_{H , O} ( t ) {X}_{H} ( t ) - ( 1 - \frac{2.86 {f}_{A , DON}}{{Y}_{A}} - \frac{4.57 {f}_{NO}}{{Y}_{A}} ) {\hat{μ}}_{A} {M}_{A , NH} ( t ) {M}_{A , O} ( t ) {X}_{A} ( t ) + ( {i}_{XB} - {f}_{I} {i}_{XP} ) {b}_{H} {M}_{H , O} ( t ) {X}_{H} ( t ) + ( {i}_{XB} - {f}_{I} {i}_{XP} ) {b}_{A} {M}_{A , O} ( t ) {X}_{A} ( t ) & ( 7 )\end{matrix}$$

where M_{H,NH}(t) is a Monod term determined by the substrate for the heterotrophic bacteria; M_{A,NH}(t) is a Monod term determined by the substrate for the autotrophic bacteria; M_{H,O}(t) is a Monod term determined by the dissolved oxygen for the heterotrophic bacteria; M_{A,O}(t) is a Monod term determined by the dissolved oxygen for the autotrophic bacteria; M_{H,DON}(t) is a Monod term determined by the mDON in the heterotrophic bacteria; k_{L}α is an exchange rate between the gas phase and the liquid phase; S_{O}* is the maximum solubility of oxygen.

The mDON in the wastewater is calculated using the following kinetic equation:

$$\frac{{dS}_{DON}}{dt} = \frac{{f}_{H , DON}}{{Y}_{H}} {\hat{μ}}_{H} {M}_{H , NH} ( t ) {M}_{H , O} ( t ) {X}_{H} ( t ) + \frac{{f}_{A , DON}}{{Y}_{A}} {\hat{μ}}_{A} {M}_{A , NH} ( t ) {M}_{A , O} ( t ) {X}_{A} ( t ) - {k}_{a} {M}_{H , DON} ( t ) {X}_{H} ( t ) \qquad ( 8 )$$

The single-step size of the AMS-mDON model is 0.1, and the total response time for the predictive model is the product of the calculation capacity and the single-step size.

The disclosure also provides a method for predicting a concentration of mDON in wastewater, the method comprising:
1) building the ASM-mDON model;
2) determining the components of an influent and the parameters of the ASM-mDON model: filtering influent samples from a wastewater treatment plant using a membrane filter; measuring the chemical oxygen demand (COD), concentrations of total nitrogen, nitrate nitrogen, nitrite nitrogen, ammonia nitrogen, and dissolved organic nitrogen of the filtered influent samples, respectively; and measuring yield coefficient Y_{H} of heterotrophic bacteria, attenuation coefficient b_{H} heterotrophic bacteria, and maximum specific growth rate \hat{μ}_{H} of heterotrophic bacteria for the activated sludge; and
3) predicting the concentration of the mDON in wastewater: inputting the components and parameters obtained in 2) into the ASM-mDON model to estimate the concentration of the mDON in the wastewater.

In 2), the wastewater treatment plant operates at the ambient temperature ranging from 15 to 25° C., and an influent pH thereof is 6.0-8.0.

In 2), the membrane filter is a cellulose acetate membrane filter having pore size of 0.45 μm.

In 2), the initial values of parameter variables are determined with reference to “Mathematical Model of Activated Sludge”. The kinetic and stoichiometric parameters of the ASM-mDON model are classified for parameter assumption, parameter estimation, or default argument assignment.

In 2), the concentration of the dissolved organic nitrogen is the difference between the total nitrogen and ammonia nitrogen, nitrate nitrogen and nitrite nitrogen; the concentration of total nitrogen is measured by using potassium persulfate oxidation-ion chromatography, or potassium persulfate oxidation-ultraviolet spectrophotometry; the concentration of ammonia nitrogen is measured by using salicylic acid-hypochlorite spectrophotometry; the nitrate nitrogen is measured by using the ion chromatography or ultraviolet-visible spectrophotometry; the nitrite nitrogen is measured by using ion chromatography or N-(1-naphthyl)-ethylenediamine spectrophotometry; and the COD is measured by using potassium dichromate method or rapid digestion method.

The following advantages are associated with the method for building a predictive model of mDON in wastewater in accordance with the disclosure:

(1) The ASM-mDON model can predict the concentration of the mDON in the wastewater, distinguish the mDON from the inDON, and quantify the concentration of the mDON released from the activated sludge in the wastewater treatment plant.

(2) The method uses a simplified ASM model, and necessary kinetic and stoichiometric parameters to build an ASM-mDON model for predicting the concentration of the mDON in the wastewater, which simplifies the operations and improves the prediction accuracy.

(3) The method can be widely applied in simulating and predicting the concentration of the mDON, laying the foundation for optimization of water quality in the wastewater treatment plants.

### BRIEF DESCRIPTION OF THE DRAWINGS

FIG. 1 is a flow chart of a method for building a predictive model of mDON according to one embodiment of the disclosure;

FIG. 2 is a graft showing the predictive result of the concentration of the mDON according to one embodiment of the disclosure; and

FIG. 3 is a graft showing the predictive result of the concentration of the mDON according to verification embodiment of the disclosure.

### DETAILED DESCRIPTION

To further illustrate the disclosure, embodiments detailing a method for building a predictive model of mDON in wastewater are described below. It should be noted that the following embodiments are intended to describe and not to limit the disclosure.

### Example 1

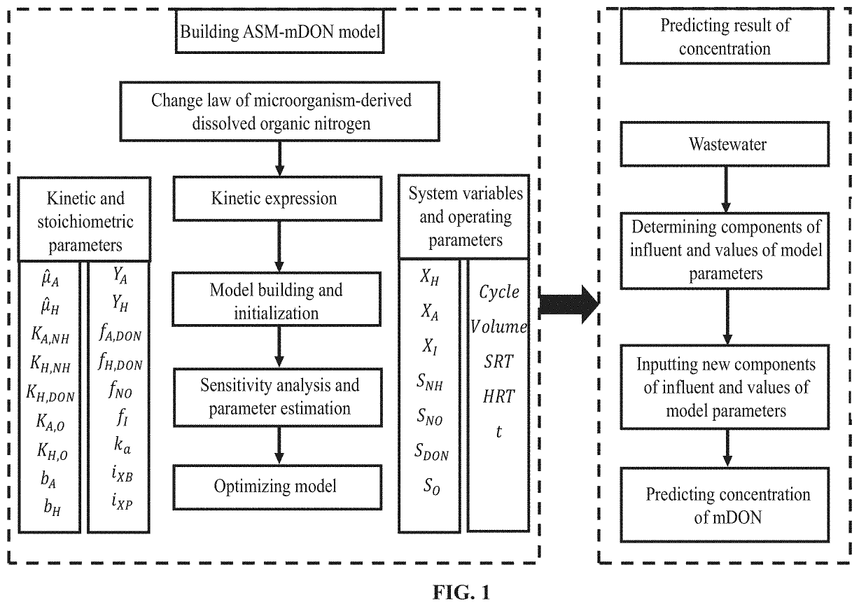

The example was a simulation of the operation of a laboratory-scale sequencing batch reactor (SBR) for the treatment of activated sludge. As a raw material, the wastewater containing particular compositions (excluding dissolved organic nitrogen) was prepared to support the growth of microorganisms in the activated sludge. The prepared wastewater contained the following compositions: 300±30 mg/L COD, 20±5 mg/L total nitrogen, and 3.5±0.5 mg/L total phosphorus. The operating parameters of the sequencing batch reactor: the effective volume of 2 L, the operating cycle of 6 h, and the hydraulic retention time of 12 h, and the activated sludge age of 20 d. The operating mode of the sequencing batch reactor at ambient temperature of 25° C.: the inlet valve opens and the influent was filled in, followed by mixing and aeration for 300 min. The mixed liquor was sedimented for 50 min and the supernatant was drained out of the sequencing batch reactor. The activated sludge in the sequencing batch reactor had a concentration within the range of 2000-200 mg/L, and a pH value of 7.5±0.5. Referring to FIG. 1, a method for building a predictive model of mDON in wastewater comprises:

1. Building the ASM-mDON Model:

The inputs to the ASM-mDON model comprises: X_{H} (concentration of heterotrophic bacteria), X_{A} (concentration of autotrophic bacteria), X_{I} (concentration of inert particles), S_{NO} (concentration of nitrate nitrogen), S_{NH} (concentration of ammonia nitrogen), S_{DON} (concentration of the mDON), S_{O} (concentration of dissolved oxygen), which were state variables; the model matrix corresponding to the process rate equation for the components were inputted into the reaction process included in the software, thereby building a simulation of the sequencing batch reactor for the treatment of activated sludge.

The simulation model was the simplified ASM-mDON model as shown in Table 1:

TABLE 1
Process rate equations for components

| Components | Process rate equation | No. |
|---|---|---|
| X_{H} | $\frac{d {X}_{H}}{d t} = {\hat{μ}}_{H} {M}_{H , NH} ( t ) {M}_{H , O} ( t ) {X}_{H} ( t ) - {b}_{H} {M}_{H , O} ( t ) {X}_{H} ( t )$ | (1) |
| X_{A} | $\frac{d {X}_{A}}{d t} = {\hat{μ}}_{A} {M}_{A , NH} ( t ) {M}_{A , O} ( t ) {X}_{A} ( t ) - {b}_{A} {M}_{A , O} ( t ) {X}_{A} ( t )$ | (2) |
| S_{NH} | $\frac{{dS}_{NH}}{dt} = - ( \frac{{f}_{H , DON}}{{Y}_{H}} + {i}_{XB} ) {\hat{μ}}_{H} {M}_{H , NH} ( t ) {M}_{H , O} ( t ) {X}_{H} ( t ) - ( \frac{{f}_{A , DON} + {f}_{NO}}{{Y}_{A}} + {i}_{XB} ) {\hat{μ}}_{A} {M}_{A , NH} ( t ) {M}_{A , O} ( t ) {X}_{A} ( t ) + {k}_{a} {M}_{H , DON} ( t ) {X}_{H} ( t )$ | (3) |
| S_{DON} | $\frac{{dS}_{DON}}{dt} = \frac{{f}_{H , DON}}{{Y}_{H}} {\hat{μ}}_{H} {M}_{H , NH} ( t ) {M}_{H , O} ( t ) {X}_{H} ( t ) + \frac{{f}_{A , DON}}{{Y}_{A}} {\hat{μ}}_{A} {M}_{A , NH} ( t ) {M}_{A , O} ( t ) {X}_{A} ( t ) - {k}_{a} {M}_{H , DON} ( t ) {X}_{H} ( t )$ | (4) |
| S_{NO} | $\frac{{dS}_{NO}}{dt} = \frac{{f}_{NO}}{{Y}_{A}} {\hat{μ}}_{A} {M}_{A , NH} ( t ) {M}_{A , O} ( t ) {X}_{A} ( t )$ | (5) |
| X_{I} | $\frac{d {X}_{I}}{d t} = {f}_{I} {b}_{H} {M}_{H , O} ( t ) {X}_{H} ( t ) + {f}_{I} {b}_{A} {M}_{A , O} ( t ) {X}_{A} ( t )$ | (6) |
| S_{O} | $\frac{{dS}_{O}}{d t} = {k}_{L} α ( {S}_{O}^{*} - {S}_{O} ) - ( 1 - \frac{2.86 {f}_{H , DON}}{{Y}_{H}} ) {\hat{μ}}_{H} {M}_{H , NH} ( t ) {M}_{H , O} ( t ) {X}_{H} ( t ) - \\ ( 1 - \frac{2.86 {f}_{A , DON}}{{Y}_{A}} - \frac{4.57 {f}_{NO}}{{Y}_{A}} ) {\hat{μ}}_{A} {M}_{A , NH} ( t ) {M}_{A , O} ( t ) {X}_{A} ( t ) + \\ ( {i}_{XB} - {f}_{I} {i}_{XP} ) {b}_{H} {M}_{H , O} ( t ) {X}_{H} ( t ) + ( {i}_{XB} - {f}_{I} {i}_{XP} ) {b}_{A} {M}_{A , O} ( t ) {X}_{A} ( t )$ | (7) |

where M_{H,NM}(t) is the Monod term determined by the substrate for the heterotrophic bacteria; M_{A,NH}(t) is the Monod term determined by the substrate for the autotrophic bacteria; M_{H,O}(t) is the Monod term determined by the dissolved oxygen for the heterotrophic bacteria; M_{A,O}(t) is the Monod term determined by the dissolved oxygen for the autotrophic bacteria; M_{H,DON}(t) is the Monod term determined by the mDON in the heterotrophic bacteria; k_{L}α is the exchange rate between the gas phase and the liquid phase; S_{O}^{*} is the maximum solubility of oxygen.

The model matrix corresponding to the process rate equation for the components were shown in Table 2:

TABLE 2
Matrix imported into reaction process included in software

| Reaction | | | | | | | | |
|---|---|---|---|---|---|---|---|---|
| process | X_{H} | X_{A} | S_{NH} | S_{DON} | S_{NO} | X_{I} | S_{O} | Process rate equation |
| Growth of heterotrophic | 1 | | $- \frac{{f}_{H , DON}}{{Y}_{H}} - {i}_{XB}$ | $\frac{{f}_{H , DON}}{{Y}_{H}}$ | | | $1 - \frac{2.86 {f}_{H , DON}}{{Y}_{H}}$ | \hat{μ}_{H}M_{H,NH}(t)M_{H,O}(t)X_{H}(t) |
| bacteria | | | | | | | | |
| Growth of autotrophic | | 1 | $- \frac{{f}_{A , DON} + {f}_{I}}{{Y}_{A}} - {i}_{XB}$ | $\frac{{f}_{A , DON}}{{Y}_{A}}$ | $\frac{{f}_{NO}}{{Y}_{A}}$ | | $1 - \frac{2.86 {f}_{A , DON}}{{Y}_{A}} - \frac{4.57 {f}_{NO}}{{Y}_{A}}$ | \hat{μ}_{A}M_{A,NH}(t)M_{A,O}(t)X_{A}(t) |
| bacteria | | | | | | | | |
| Endogenous | −1 | | | | | f_{I} | | b_{H}M_{H,O}(t)X_{H}(t) |
| respiration of | | | | | | | | |
| heterotrophic | | | | | | | | |
| bacteria | | | | | | | | |
| Endogenous | | −1 | | | | f_{I} | | b_{A}M_{A,O}(t)X_{A}(t) |
| respiration of | | | | | | | | |
| autotrophic | | | | | | | | |
| bacteria | | | | | | | | |
| Arnmonization | | | 1 | −1 | | | | kₐM_{H,DON}(t)X_{H}(t) |

2. Determining the components of influent and the values of model parameters of the ASM-mDON model:

50 mL of influent was sampled directly from the sequencing batch reactor, and filtered using a cellulose acetate membrane filter having pore size of 0.45 μm. The concentration of total nitrogen was 20 mg/L measured by using potassium persulfate oxidation-ion chromatography, or potassium persulfate oxidation-ultraviolet spectrophotometry; the concentration of ammonia nitrogen was 20 mg/L measured by using salicylic acid-hypochlorite spectrophotometry; the nitrate nitrogen was 0 mg/L measured by using the ion chromatography or ultraviolet-visible spectrophotometry; the nitrite nitrogen was 0 mg/L measured by using ion chromatography or N-(1-naphthyl)-ethylenediamine spectrophotometry; and the COD was 300 mg/L measured by using potassium dichromate method or rapid digestion method. The concentration of the dissolved organic nitrogen was 0 mg/L, that is, the difference between the sum of total nitrogen and ammonia nitrogen and the sum of nitrate nitrogen and nitrite nitrogen.

The default values for kinetics and stoichiometric parameters of the conventional model, and the water quality parameters determined in 1) were used for simulation of parameters in relation to the mDON yielded in the wastewater treatment process; the simulation parameters were set as follows: \hat{μ}_{H}, 0.8 h⁻¹, Y_{H}, 0.67 mg (COD)/mg (N); b_{H}, 0.62 h⁻¹; K_{H,NH}, 0.05 mg (N)/L; K_{H,O}, 0.2 mg (N)/L; \hat{μ}_{A}, 0.3 h⁻¹; f_{H,DON}, 0.04; Y_{A}, 3.4 mg (COD)/mg (N); b_{A}, 0.15 h⁻¹; K_{A,NH}, 5 mg (N)/L; K_{A,O}, 0.4 mg (N)/L; f_{A,DON}, 0.04, i_{XB}, 0.07 mg (N)/mg (COD); i_{XP}, 0.03 mg (N)/mg (COD); f_{NO}, 0.8; f_{I}, 0.2; kₐ, 0.04 L/(mg (N)·d); K_{H,DON}, 1.5 mg (N)/L; where pH was the maximum specific growth rate of heterotrophic bacteria, Y_{H} was the yield coefficient of heterotrophic bacteria, b_{H} was the attenuation coefficient of heterotrophic bacteria, K_{H,NH} was the half-saturation constant for ammonia nitrogen of heterotrophic bacteria, K_{H,O} was the half-saturation constant for dissolved oxygen of heterotrophic bacteria, \hat{μ}_{A} was the maximum specific growth rate of autotrophic bacteria, f_{H,DON} was the substrate utilization ratio of heterotrophic bacteria converting the substrates into the mDON, Y_{A} was the yield coefficient of autotrophic bacteria, b_{A} was the attenuation coefficient of autotrophic bacteria, K_{A,NH} was the half-saturation constant for ammonia nitrogen of autotrophic bacteria, K_{A,O} was the half-saturation constant for dissolved oxygen of autotrophic bacteria, f_{A,DON} was the substrate utilization ratio of autotrophic bacteria converting the substrates into the mDON, i_{XB} was the proportion of nitrogen in the organism, i_{XP} was the proportion of nitrogen in the product of the organism. f_{NO} was the substrate utilization ratio of autotrophic bacteria converting the substrates into the nitrate nitrogen, f_{I} was the rate of inert particles yielded in the organism, kₐ was the ammonification rate. K_{H,DON} was the half-saturation constant for mDON.

3. Predicting the concentration of mDON in wastewater:

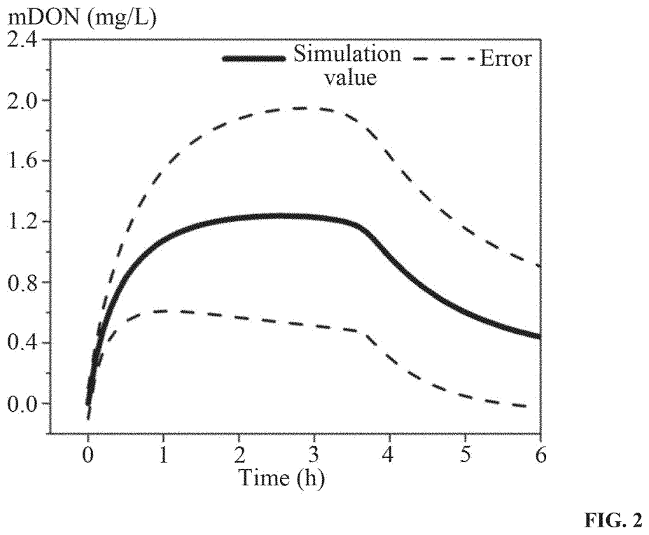

The components of influent and values of model parameters determined in 2) were fed into the software for modeling mDON to predict the concentration of the mDON in wastewater; where the single-step size was 0.1, and the calculation capacity was 60 steps, and the simulation process was based on the mDON participating in the biochemical reactions. The model-predicted result was shown in FIG. 2.

### Example 2

The example was the same as Example 1, except for the influent from the municipal wastewater treatment plant A. The operating parameters of the sequencing batch reactor: the influent temperature was 15° C., and hydraulic retention time was 8 h, activated sludge age was 20 d. The influent contains the following compositions: COD 96.2-120.6 mg/L, total nitrogen 23.7-29.1 mg/L, total phosphorus 2.0-3.5 mg/L, pH 7.4-8.0, and inert particles 3000-3200 mg/L. The influent (of greater than 200 mL) in the biological treatment process (i.e. oxidation ditch) and the activated sludge (of greater than 50 mL) were sampled for analysis of the components of the influent, as well as parameter estimation. The influent sample was then filtered using a cellulose acetate membrane filter having pore size of 0.45 μm. The COD was measured by using potassium dichromate method or rapid digestion method; the concentration of total nitrogen was measured by using potassium persulfate oxidation-ion chromatography, or potassium persulfate oxidation-ultraviolet spectrophotometry; the concentration of ammonia nitrogen was measured by using salicylic acid-hypochlorite spectrophotometry; the nitrate nitrogen was measured by using the ion chromatography or ultraviolet-visible spectrophotometry; the nitrite nitrogen was measured by using ion chromatography or N-(1-naphthyl)-ethylenediamine spectrophotometry; the concentration of dissolved organic nitrogen was the difference between the sum of total nitrogen and ammonia nitrogen and the sum of nitrate nitrogen and nitrite nitrogen. According to the parameters determined in 1), the initial values of the yield coefficient (Y_{H}) of heterotrophic bacteria, the attenuation coefficient of heterotrophic bacteria, and the maximum specific growth rate (\hat{μ}_{H}) of heterotrophic bacteria were 0.26 mgCOD/mgN, 0.09 h⁻¹, and 1.0 h⁻¹, respectively.

Building the ASM-mDON model and finding the optimal parameter values i_{XB} by parameter estimation: 0.07 mg (N)/mg (COD); kₐ, 0.04 L/(mg (N)·d); \hat{μ}_{H}, 1.0 h⁻¹; Y_{H}, 0.30 mg (COD)/mg (N); b_{H}, 0.05 h⁻¹; K_{H,NH}, 0.05 mg (N)/L; K_{H,O}, 0.2 mg (N)/L; \hat{μ}_{A}, 0.3 h⁻¹; f_{H,DON}, 0.04; Y_{A}, 3.4 mg (COD)/mg (N); b_{A}, 0.15 h⁻¹; K_{A,NH}, 5 mg (N)/L; K_{A,O}, 0.4 mg (N)/L; f_{A,DON}, 0.04; i_{XP}, 0.03 mg (N)/mg (COD); f_{NO}, 0.8; f_{I}, 0.2; K_{H,DON}, 1.5 mg (N)/L.

Predicting the concentration of the mDON in wastewater: the components of influent and values of model parameters determined in 2) were fed into the software for modeling mDON to predict the concentration of the mDON in wastewater; where the single-step size was 0.1, and the calculation capacity was 240 steps. The model-predicted concentration of the mDON yielded in the oxidation ditch was 2.32 mg/L.

### Example 3

The example was the same as Example 2, except for the influent coming from the municipal wastewater treatment plant A was sampled at different times. The operating parameters of the sequencing batch reactor: the influent temperature was 20° C., and hydraulic retention time was 8 h, activated sludge age was 5 d. The influent contains the following compositions: COD 96.2-120.6 mg/L, total nitrogen 23.7-29.1 mg/L, total phosphorus 2.0-3.5 mg/L, pH 7.4-8.0, and inert particles 3000-3200 mg/L. The influent (of greater than 200 mL) in the biological treatment process (i.e. oxidation ditch) and the activated sludge (of greater than 50 mL) were sampled for analysis of the components of the influent and parameter estimation. The influent sample was then filtered using a cellulose acetate membrane filter having pore size of 0.45 μm. COD was measured by using potassium dichromate method or rapid digestion method; the concentration of total nitrogen was measured by using potassium persulfate oxidation-ion chromatography, or potassium persulfate oxidation-ultraviolet spectrophotometry; the concentration of ammonia nitrogen was measured by using salicylic acid-hypochlorite spectrophotometry; the nitrate nitrogen was measured by using the ion chromatography or ultraviolet-visible spectrophotometry; the nitrite nitrogen was measured by using ion chromatography or N-(1-naphthyl)-ethylenediamine spectrophotometry; the concentration of dissolved organic nitrogen was the difference between the sum of total nitrogen and ammonia nitrogen and the sum of nitrate nitrogen and nitrite nitrogen. According to the parameters determined in 1), the initial values of the yield coefficient (Y_{H}) of heterotrophic bacteria, the attenuation coefficient of heterotrophic bacteria, and the maximum specific growth rate (PH) of heterotrophic bacteria were 0.26 mgCOD/mgN, 0.09 h⁻¹, and 1.0 h⁻¹, respectively.

Building the ASM-mDON model and finding the optimal parameter values i_{XB} by parameter estimation: 0.07 mg (N)/mg (COD); kₐ, 0.04 L/(mg (N)·d); \hat{μ}_{H}, 1.0 h⁻¹; Y_{H}, 0.30 mg (COD)/mg (N); b_{H}, 0.05 h⁻¹; K_{H,NH}, 0.05 mg (N)/L; K_{H,O}, 0.2 mg (N)/L; \hat{μ}A, 0.3 h⁻¹, f_{H,DON}, 0.04; Y_{A}, 3.4 mg (COD)/mg (N); b_{A}, 0.15 h⁻¹, K_{A,NH}, 5 mg (N)/L; K_{A,O}, 0.4 mg (N)/L; f_{A,DON}, 0.04; i_{XP}, 0.03 mg (N)/mg (COD); f_{NO}, 0.8; f_{I}, 0.2; K_{H,DON}, 1.5 mg (N)/L.

Predicting the concentration of mDON in wastewater: the components of influent and values of model parameters determined in 2) were fed into the software for modeling mDON to predict the concentration of the mDON in wastewater; where the single-step size was 0.1, and the calculation capacity was 240 steps. The model-predicted concentration of the mDON yielded in the oxidation ditch was 1.89 mg/L.

### Example 4

The example was the same as Example 1, except for the influent from the municipal wastewater treatment plant B. The operating parameters of the sequencing batch reactor: the influent temperature was 20° C., and hydraulic retention time was 6 h. activated sludge age was 30 days. The influent contained the following compositions: COD 130.9 mg/L, total nitrogen 25.1 mg/L, total phosphorus 5.1 mg/L, pH 7.2, and inert particles 3000-3200 mg/L. The influent (of greater than 200 mL) in the biological treatment process (i.e. oxidation ditch) and the activated sludge (of greater than 50 mL) were sampled for analysis of the components of the influent and parameter estimation. The influent sample was then filtered using a cellulose acetate membrane filter having pore size of 0.45 μm. COD was measured by using potassium dichromate method or rapid digestion method; the concentration of total nitrogen was measured by using potassium persulfate oxidation-ion chromatography, or potassium persulfate oxidation-ultraviolet spectrophotometry; the concentration of ammonia nitrogen was measured by using salicylic acid-hypochlorite spectrophotometry; the nitrate nitrogen was measured by using the ion chromatography or ultraviolet-visible spectrophotometry; the nitrite nitrogen was measured by using ion chromatography or N-(1-naphthyl)-ethylenediamine spectrophotometry; the concentration of dissolved organic nitrogen was the difference between the sum of total nitrogen and ammonia nitrogen and the sum of nitrate nitrogen and nitrite nitrogen. According to the parameters determined in 1), the initial values of the yield coefficient (Y_{H}) of heterotrophic bacteria, the attenuation coefficient of heterotrophic bacteria, and the maximum specific growth rate (\hat{μ}_{H}) of heterotrophic bacteria were 0.2 mgCOD/mgN, 0.05 h⁻¹, and 0.3 h⁻¹, respectively.

Building the ASM-mDON model and finding the optimal parameter values i_{XB} by parameter estimation: 0.07 mg (N)/mg (COD); kₐ, 0.04 L/(mg (N)·d); \hat{μ}_{H}, 0.33 h⁻¹; Y_{H}, 0.32 mg (COD)/mg (N); b_{H}, 0.05 h⁻¹; K_{H,NH}, 0.05 mg (N)/L; K_{H,O}, 0.2 mg (N)/L; \hat{μ}_{A}, 0.3 h⁻¹; f_{H,DON}, 0.04; Y_{A}, 3.0 mg (COD)/mg (N); b_{A}, 0.15 h⁻¹; K_{A,NH}, 5 mg (N)/L; K_{A,O}, 0.4 mg (N)/L; f_{A,DON}, 0.04; i_{XP}, 0.03 mg (N)/mg (COD); f_{NO}, 0.8; f_{I}, 0.2; K_{H,DON}, 1.5 mg (N)/L.

Predicting the concentration of mDON in wastewater: the components of influent and values of model parameters determined in 2) were fed into the software for modeling mDON to predict the concentration of the mDON in wastewater; where the single-step size was 0.1, and the calculation capacity was 60 steps. The model-predicted concentration of the mDON yielded in the oxidation ditch was 4.31 mg/L.

### Verification Example

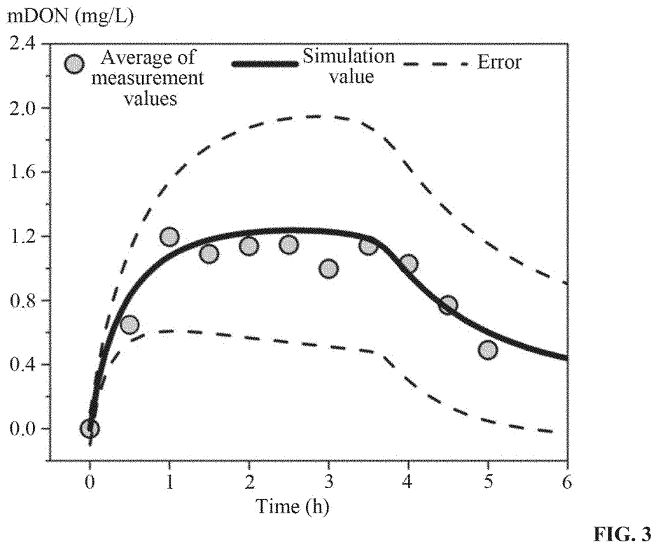

The influent entering the sequencing batch reactor in Example 1 was sampled to measure the concentration of the mDON that was then verified with the model-predicted concentration. The operating cycle for the sequencing batch reactor was 5 h, under which the influent was sampled per 0.5 h interval. The influent samples were filtered and used to undergo measurement with reference to the methods described in Example 1. Referring to FIG. 3, the measured concentration of the mDON in the entire operating cycle was basically fitted to the model-predicted concentration, lying in the calculated error range.

In summary, the measured concentration of the mDON is close to the model-predicted concentration of the mDON yielded in the activated sludge process in accordance with the Verification Example of the disclosure. The disclosure offers many advantages in simplicity, accuracy, and fast prediction over current methods, thereby being widely applied in prediction of the mDON yielded in activated sludge process.

It will be obvious to those skilled in the art that changes and modifications may be made, and therefore, the aim in the appended claims is to cover all such changes and modifications.

What is claimed is:

1. A method, comprising:
a) acquiring a kinetics associated with production and consumption of a mDON of an activated sludge system, and importing a kinetic expression of the mDON into a conventional activated sludge model No. 1 (ASM1) to build a kinetic equation for the mDON;
b) inputting component variables, parameter variables, model matrices, process rate equation and operating parameters of a predictive model into a simulation software AquaSim to build an ASM-mDON model;
c) inputting initial values of the component variables and the parameter variables into the simulation software AquaSim for model initialization;
d) acquiring initial mDON kinetic and sensitivity analysis results, selecting corresponding parameters, calibrating kinetic and stoichiometric parameters of the ASM-mDON model using a parameter estimation function of the simulation software AquaSim, thereby predicting a concentration of the mDON; and
e) replacing the initial values of the ASM-mDON model with optimal values obtained by the parameter estimation function in d), thereby optimizing the model.

2. The method of claim 1, wherein the activated sludge system comprises a fully mixed steady state activated sludge: the activated sludge has a sludge age of 5-30 days, and a concentration of 2000-5000 mg/L.

3. The method of claim 1, wherein the ASM-mDON model is used for study of the mDON released by microorganisms in the activated sludge system, and the model comprises:
seven components: heterotrophic bacteria X_{H}, autotrophic bacteria X_{A}, inert particles X_{I}, nitrate nitrogen S_{NO}, ammonia nitrogen S_{NH}, microorganism-derived dissolved organic nitrogen S_{DON}, dissolved oxygen S_{O};
five reaction processes: a growth process and an endogenous respiration process of heterotrophic bacteria using ammonium chloride as a substrate; a growth process and an endogenous respiration process of autotrophic bacteria using ammonium chloride as a substrate; and an ammonization process of mDON; and
eighteen parameters: maximum specific growth rate \hat{μ}_{H} of heterotrophic bacteria, yield coefficient Y_{H} of heterotrophic bacteria, attenuation coefficient b_{H} of heterotrophic bacteria, half-saturation constant K_{H,NH} for ammonia nitrogen of heterotrophic bacteria, half-saturation constant K_{H,O} for dissolved oxygen of heterotrophic bacteria, maximum specific growth rate \hat{μ}_{A} of autotrophic bacteria, substrate utilization ratio f_{H,DON} of heterotrophic bacteria converting the substrate into the mDON, yield coefficient Y_{A} of autotrophic bacteria, attenuation coefficient b_{A} of autotrophic bacteria, half-saturation constant K_{A,NH} for ammonia nitrogen of autotrophic bacteria, half-saturation constant K_{A,O} for dissolved oxygen of autotrophic bacteria, substrate utilization ratio f_{A,DON} of autotrophic bacteria converting the substrate into the mDON, proportion of nitrogen i_{XB} in an organism, proportion of nitrogen i_{XP} in the product of the organism, substrate utilization ratio f_{NO} of autotrophic bacteria converting the substrate into the nitrate nitrogen, proportion of inert particles f_{I} yielded in the organism, ammonification rate kₐ, and half-saturation constant K_{H,DON} for mDON.

4. The method of claim 3, wherein change rates of the seven components of the ASM-mDON model satisfy with the following formulas:
$$\begin{matrix}{X}_{H} \text{:} \frac{{dX}_{H}}{dt} = {\hat{μ}}_{H} {M}_{H , NH} ( t ) {M}_{H , O} ( t ) {X}_{H} ( t ) - {b}_{H} {M}_{H , O} ( t ) {X}_{H} ( t ) & ( 1 ) \\ {X}_{A} \text{:} \frac{{dX}_{A}}{dt} = {\hat{μ}}_{A} {M}_{A , NH} ( t ) {M}_{A , O} ( t ) {X}_{A} ( t ) - {b}_{A} {M}_{A , O} ( t ) {X}_{A} ( t ) & ( 2 ) \\ {S}_{NH} \text{:} \frac{{dS}_{NH}}{dt} = - ( \frac{{f}_{H , DON}}{{Y}_{H}} + {i}_{XB} ) {\hat{μ}}_{H} {M}_{H , NH} ( t ) {M}_{H , O} ( t ) {X}_{H} ( t ) - ( \frac{{f}_{A , DON} + {f}_{NO}}{{Y}_{A}} + {i}_{XB} ) {\hat{μ}}_{A} {M}_{A , NH} ( t ) {M}_{A , O} ( t ) {X}_{A} ( t ) + {k}_{a} {M}_{H , DON} ( t ) {X}_{H} ( t ) & ( 3 ) \\ {S}_{DON} \text{:} \frac{{dS}_{DON}}{dt} = \frac{{f}_{H , DON}}{{Y}_{H}} {\hat{μ}}_{H} {M}_{H , NH} ( t ) {M}_{H , O} ( t ) {X}_{H} ( t ) + \frac{{f}_{A , DON}}{{Y}_{A}} {\hat{μ}}_{A} {M}_{A , NH} ( t ) {M}_{A , O} ( t ) {X}_{A} ( t ) - {k}_{a} {M}_{H , DON} ( t ) {X}_{H} ( t ) & ( 4 ) \\ {S}_{NO} \text{:} \frac{{dS}_{NO}}{dt} = \frac{{f}_{NO}}{{Y}_{A}} {\hat{μ}}_{A} {M}_{A , NH} ( t ) {M}_{A , O} ( t ) {X}_{A} ( t ) & ( 5 ) \\ {X}_{I} \text{:} \frac{{dX}_{I}}{dt} = {f}_{I} {b}_{H} {M}_{H , O} ( t ) {X}_{H} ( t ) + {f}_{I} {b}_{A} {M}_{A , O} ( t ) {X}_{A} ( t ) & ( 6 ) \\ {S}_{O} \text{:} \frac{{dS}_{O}}{dt} = {k}_{L} α ( {S}_{O}^{*} - {S}_{O} ) - ( 1 - \frac{2.86 {f}_{H , DON}}{{Y}_{H}} ) {\hat{μ}}_{H} {M}_{H , NH} ( t ) {M}_{H , O} ( t ) {X}_{H} ( t ) - ( 1 - \frac{2.86 {f}_{A , DON}}{{Y}_{A}} - \frac{4.57 {f}_{NO}}{{Y}_{A}} ) {\hat{μ}}_{A} {M}_{A , NH} ( t ) {M}_{A , O} ( t ) {X}_{A} ( t ) + ( {i}_{XB} - {f}_{I} {i}_{XP} ) {b}_{H} {M}_{H , O} ( t ) {X}_{H} ( t ) + ( {i}_{XB} - {f}_{I} {i}_{XP} ) {b}_{A} {M}_{A , O} ( t ) {X}_{A} ( t ) & ( 7 )\end{matrix}$$
M_{H,NH}(t) is a Monod term determined by the substrate for the heterotrophic bacteria: M_{A,NH}(t) is a Monod term determined by the substrate for the autotrophic bacteria; M_{H,O}(t) is a Monod term determined by the dissolved oxygen for the heterotrophic bacteria; M_{A,O}(t) is a Monod term determined by the dissolved oxygen for the autotrophic bacteria; M_{H,DON}(t) is a Monod term determined by the mDON in the heterotrophic bacteria: k_{L}α is an exchange rate between a gas phase and a liquid phase; and S_{O}* is a maximum solubility of oxygen.

5. The method of claim 3, wherein the mDON in wastewater is calculated using the following kinetic equation:
$$\frac{{dS}_{DON}}{dt} = \frac{{f}_{H , DON}}{{Y}_{H}} {\hat{μ}}_{H} {M}_{H , NH} ( t ) {M}_{H , O} ( t ) {X}_{H} ( t ) + \frac{{f}_{A , DON}}{{Y}_{A}} {\hat{μ}}_{A} {M}_{A , NH} ( t ) {M}_{A , O} ( t ) {X}_{A} ( t ) - {k}_{a} {M}_{H , DON} ( t ) {X}_{H} ( t ) . \qquad ( 8 )$$

6. The method of claim 3, wherein a single-step size of the AMS-mDON model is 0.1, and a total response time for the predictive model is a product of a calculation capacity and the single-step size.

7. A method for predicting a concentration of mDON in wastewater, the method comprising:
1) building the ASM-mDON model according to the method of claim 1;
2) determining components of an influent and the parameters of the ASM-mDON model, comprising: filtering an influent sample from a wastewater treatment plant using a membrane filter; measuring chemical oxygen demand (COD), concentrations of total nitrogen, nitrate nitrogen, nitrite nitrogen, ammonia nitrogen, and dissolved organic nitrogen of the influent sample filtered, respectively; and measuring yield coefficient Y_{H} of heterotrophic bacteria, attenuation coefficient b_{H} heterotrophic bacteria, and maximum specific growth rate \hat{μ}_{H} of heterotrophic bacteria for the activated sludge; and
3) predicting the concentration of the mDON in wastewater, comprising: inputting the components and parameters obtained in 2) into the ASM-mDON model to estimate the concentration of the mDON in the wastewater.

8. The method of claim 7, wherein in 2), the wastewater treatment plant operates at an ambient temperature ranging from 15 to 25° C., and an influent pH thereof is 6.0-8.0.

9. The method of claim 7, wherein in 2), the concentration of the dissolved organic nitrogen is a difference between concentrations of total nitrogen and ammonia nitrogen, nitrate nitrogen and nitrite nitrogen; the concentration of the total nitrogen is measured by using potassium persulfate oxidation-ion chromatography, or potassium persulfate oxidation-ultraviolet spectrophotometry; the concentration of the ammonia nitrogen is measured by using salicylic acid-hypochlorite spectrophotometry; the concentration of the nitrate nitrogen is measured by using the ion chromatography or ultraviolet-visible spectrophotometry; the concentration of the nitrite nitrogen is measured by using ion chromatography or N-(1-naphthyl)-ethylenediamine spectrophotometry; and the COD is measured by using potassium dichromate method or rapid digestion method.
